(19) United States
(12) Patent Application Publication
(10) Pub. No.: US 2018/0339135 A1
(43) Pub. Date: Nov. 29, 2018

(54) CANNULA SECURING DEVICE

(71) Applicant: Menachem Nathan, Tel Aviv (IL)

(72) Inventor: Menachem Nathan, Tel Aviv (IL)

(21) Appl. No.: 15/975,053

(22) Filed: May 9, 2018

Related U.S. Application Data
(60) Provisional application No. 62/512,012, filed on May 28, 2017.

(51) Int. Cl. A61M 25/02; A61M 25/06

(52) U.S. Cl. CPC A61M 25/02; A61M 2005/1586; A61M 25/0637; A61M 25/0606

(57) ABSTRACT

Cannula securing devices and cannula needle and catheter covers including such devices. wherein a cannula securing device comprises a tubular section shaped internally to engage and hold securely a cannula needle bushing, the tubular section having a top and a bottom, and a securing member for attaching the securing device to a patient's skin in a substantially perpendicular orientation to the skin, wherein the securing device has an attachment surface substantially co-planar with the tubular section bottom.

### CROSS REFERENCE TO RELATED APPLICATIONS

This application is related to, and claims priority from U.S. Provisional Patent Application No. 62/512,012 filed May 28, 2017 and having the same title, which is incorporated herein by reference in its entirety.

### FIELD

Embodiments disclosed herein relate in general to devices for draining fluids from a body cavity such as a cannula (also referred to as IV cannula or IV catheter) and in particular to devices for securing a cannula to a patient's external body surface (i.e. skin) during, for example, thoracentesis or paracentesis.

### BACKGROUND

IV cannulae are known. FIG. 1 shows a known art cannula 10 that includes a catheter assembly 12 having a catheter 14 with a proximal end and a distal end. As used herein, the term “proximal” refers to a location on the device that is closest to the clinician using the device and farthest from the patient on whom the device is used in its normal operation. Conversely, the term “distal” refers to a location on the device that is farthest from the clinician using the device and closest to the patient. The catheter includes an introducer needle 16 having a sharp distal tip defined by a bevel 18. Needle 16 is held at its proximal end in a bushing 20, which may be formed integrally with or attached to a catheter hub 22 with two wings 24. Suitable materials for catheter 14 include, but are not limited to, thermoplastic resins such as polytetrafluoroethylene (PTFE), fluorinated ethylene propylene copolymer (FEP), polyurethane and the like. Preferably, catheter 14 is formed from thermoplastic hydrophilic polyurethane that softens with exposure to physiological conditions present in the patient's body.

Cannula 10 may further include a radially outwardly extending side port 26, which is useful for connecting a fluid handling device to catheter 14 for infusing fluids into the patient's blood vessel. Preferably, side port 26 extends upwardly away from the patient when catheter 14 is inserted into the patient. Wings 24 are useful to stabilize catheter 14 in the patient and provide a surface that facilitates taping of catheter assembly 12 to the patient to fix catheter 14 properly in the patient's vasculature. When fixed to the patient, wings 24 are substantially parallel to, or inclined at a very shallow angle to the patient's skin and vasculature. Therefore, in this description, they are also referred to as “parallel wings”. Note that wings 24 have grooves (thinned linear section) 28 that enable to wings to be bent upwards for convenient packaging.

A needle and catheter cover (or simply “cover”) 30 may be used to cover catheter 14 and introducer needle 16 prior to use. Cover 30 has a proximal end 32 that can fit snugly over bushing 20, being held in place by friction. Preferably cover 30 is formed from hard polymeric materials such as thermoplastic polymeric resins, which include polycarbonate, polystyrene, polypropylene and the like. Of course other materials may also be used for cover 30.

In both paracentesis and thoracentesis, the catheter is inserted into a body organ at substantially 90 degrees (substantially perpendicular) to the external surface (i.e. skin) of the body organ through which it was inserted. In most (if not all) cases, the introducer needle is inserted until the bushing touches the skin surface. Once the catheter is inserted during thoracentesis, it must remain fixed in the substantially perpendicular orientation to the body organ. The same orientation is preferable in paracentesis and may be useful in other procedures for draining fluids from the body. In addition, the catheter and cannula must be secured in place, since if unsecured, both may move or even dislodge from the body.

Suggested attempts to provide simple securing devices that can hold and secure a catheter or cannula to the body in a substantially perpendicular orientation remain unsatisfactory. Such devices include for example those disclosed in European patent EP0232600 B1, U.S. Pat. No. 5,897,531, US patent application 20140039453 and international patent application PCT/US2014/058456. A major disadvantage of the device proposed in EP EP0232600 B1 lies in the relatively large number of added parts/features added to the ones existing in a simple cannula, which increase the size and cost of the cannula and complicate its use. These added parts include (but are not limited to) a flange, an axially extending hub, an annular locking member or collet, a four-section jaw and a recess. The other proposed solutions suffer from similar and other disadvantages.

There is therefore a need for, and it would be advantageous to have, a simple-to-use device with simple design and fewer parts than known securing devices, to hold a catheter and/or cannula secured to the body organ in an orientation substantially perpendicular to the body organ.

### SUMMARY

Aspects of embodiments disclosed herein relate to cannula securing devices and more specifically to cannula holders that secure a cannula and/or catheter to skin in a substantially perpendicular orientation. As used herein, “substantially perpendicular” refers to a truly perpendicular (i.e. 90 degrees) orientation to a surface, as well as to an orientation that deviates for the truly perpendicular by a few degrees, i.e. by 1, 2, 3, 4, 5 and even 10 degrees.

Aspects of embodiments disclosed herein also relate to catheter and introducer needle covers that include two parts, wherein one part is structured and adapted to serve as a cannula holder. As used herein, the term “cannula holder cover” includes also needle covers, catheter covers and needle-catheter assembly covers.

In exemplary embodiments, there are provided cannula securing devices comprising a tubular section having a bottom and shaped internally to engage and hold securely a cannula needle bushing, and a securing member for attaching the cannula securing device to a patient's skin via a contact (attachment) surface in an orientation substantially perpendicular to the skin, wherein the attachment surface is substantially co-planar with the tubular section bottom.

In an exemplary embodiment of a cannula securing device, the securing member includes at least one perpendicular securing wing (or simply “perpendicular wing”) and the attachment surface is part of the at least one perpendicular wing. In some embodiments, the securing member may include two perpendicular wings or four perpendicular rings. Each perpendicular wing may include a groove allowing it to be bent or folded. In other exemplary embodiments, the securing member may include a disc or a plate. The securing member may be made of hard polymeric materials such as thermoplastic polymeric resins, which include polycarbonate, polystyrene, polypropylene and the like.

In exemplary embodiments, there are provided cannula needle and catheter covers (also referred to simply as “covers”) comprising a proximal section shaped to serve as a cannula securing device and including a tubular section having a bottom and shaped internally to engage and hold securely a cannula needle bushing, and a securing member for attaching the cannula securing device to a patient's skin in an orientation substantially perpendicular to the skin, and a distal section connectable to, and disconnectable from the proximal section.

In some exemplary embodiments of a cover, the securing member includes at least one perpendicular wing and the attachment surface is part of the at least one perpendicular wing. In some embodiments, the securing member may include two perpendicular wings or four perpendicular rings. Each perpendicular wing may include a groove allowing it to be bent or folded. In other exemplary embodiments, the securing member may include a disc or a plate. The securing member may be made of hard polymeric materials such as thermoplastic polymeric resins, which include polycarbonate, polystyrene, polypropylene and the like.

In an exemplary embodiment, there is provided a method for securing a cannula and/or catheter to a body organ, comprising providing a cannula securing device that includes a tubular section having a bottom, the tubular section shaped internally to engage and hold securely a cannula needle bushing, and a securing member having an attachment surface substantially co-planar with the tubular section bottom, attaching the cannula securing device to a patient's skin via the attachment surface, and inserting the cannula into the body organ through the tubular section such that the cannula needle bushing is engaged and securely held in the tubular section. In an embodiment of the method, the attaching includes attaching the cannula securing device to the patient's skin in an orientation substantially perpendicular to the skin

### BRIEF DESCRIPTION OF THE DRAWINGS

In order to better understand the subject matter disclosed herein and to exemplify how it may be carried out in practice, embodiments will now be described, by way of non-limiting example only, with reference to the accompanying drawings, in which:

FIG. 1 shows a known art cannula;

FIG. 2 shows an embodiment of a cannula holder with four perpendicular securing wings in (a) an isomeric view from a bottom side, in (b) an isomeric view from a top side, and (c) a side view;

FIG. 3 shows embodiments of cannula holders with (a) one perpendicular securing wing, and (b) four perpendicular securing wings;

FIG. 4 shows an embodiment of a needle and catheter cover with an integrated cannula holder in (a) an isomeric view, (b) a side view, (c) a side cross section along a longitudinal axis of the needle and catheter, and (d) a side cross section in which the two parts of the cover are shown separated;

FIG. 5 shows an embodiment of a two-part cannula catheter cover, in which the perpendicular securing wings of a holder are (a) deployed for attachment, (b) folded forward toward the distal end of the catheter and (c) folded backward toward the proximal end of the catheter;

FIG. 6 shows in (a) a cannula holder disclosed herein attached to a body organ with a catheter and needle partially inserted into the body organ, and in (b) the catheter and cannula secured to the body through the cannula holder.

### DETAILED DESCRIPTION

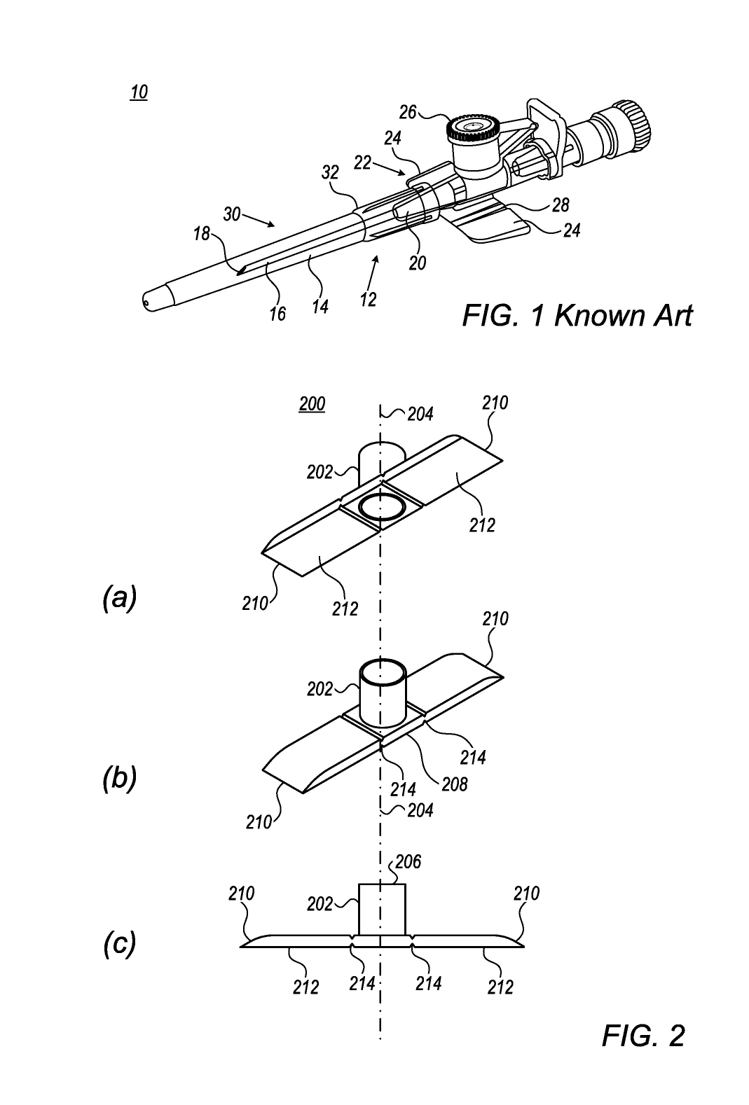

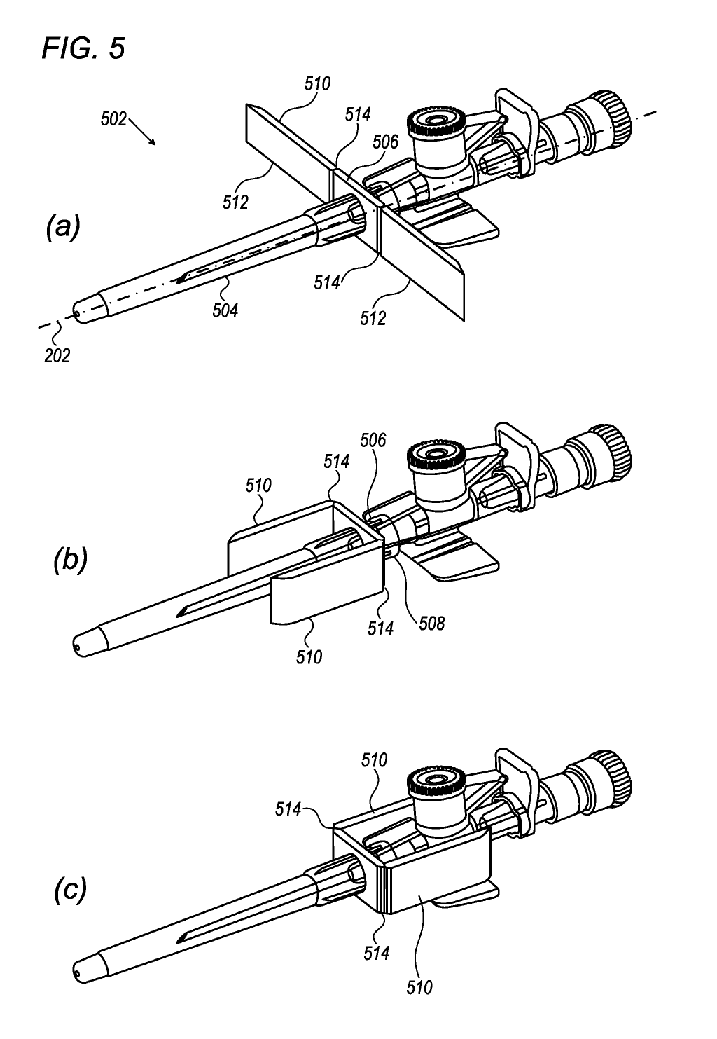

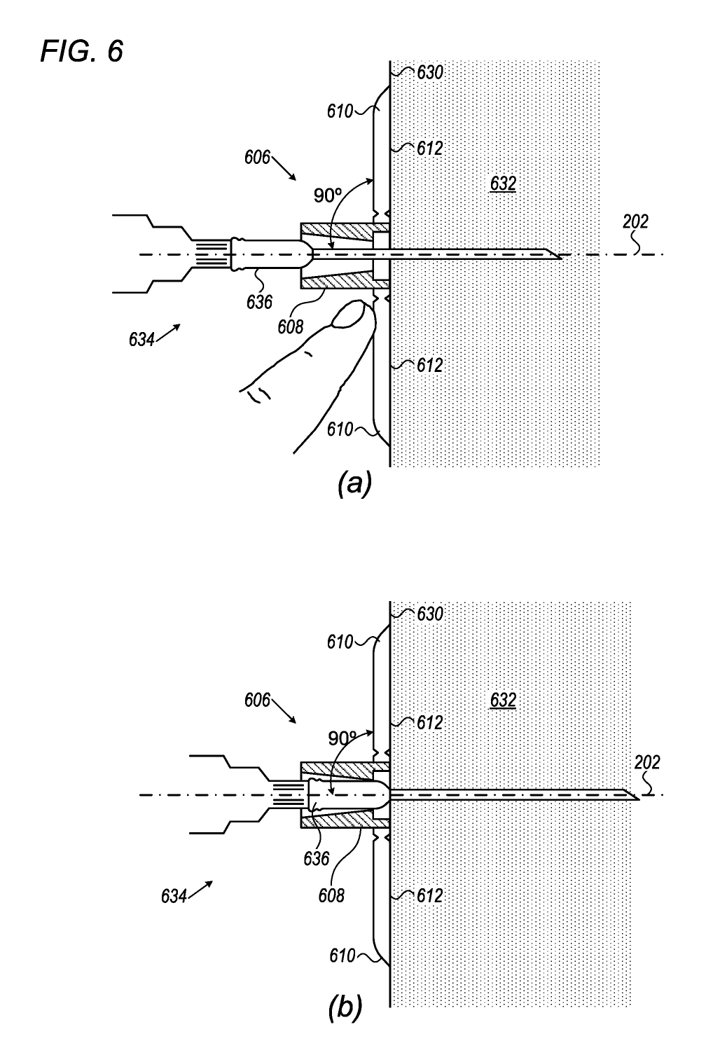

FIG. 2 shows an embodiment numbered 200 of a cannula securing device (also referred to as “cannula holder”) disclosed herein. Cannula holder 200 includes a generally tubular section (or “tube”) 202 with a longitudinal axis 204, tube 202 shaped to engage and accommodate a needle bushing such as bushing 20 in FIG. 1. Tube 202 has a top side 206 and a bottom side 208. Cannula holder 200 further includes a securing member in the form of a pair of securing wings 210 fixedly attached to tube 202 and lying in a plane generally perpendicular to axis 204. Accordingly, wings 210 are also referred to throughout this description as “perpendicular wings”. The term “securing wings” indicate the function of wings 210 in securing cannula holder 200 to a patient's body, for example a patient's abdomen in case of paracentesis or a patient's back in case of thoracentesis (see FIG. 6). In use, wings 210 contact the skin (or are attachable to the skin) with respective contact surfaces 212. Contact surfaces 212 are substantially co-planar with bottom side 208 of tube 202. Similar to parallel wings 24, securing wings 210 may include thinned materials sections (e.g. grooves) 214 which allow wings 210 to be bent or folded along axis 204 in a forward, backward or both forward and backward directions (see respectively FIG. 5 (b) and (c)).

Advantageously, tube 202 is shaped internally to accommodate needle bushing 20 with some degree of friction. For example, the degree of friction may be that normally present between needle bushings and covers in regular cannulae. For example, the degree of friction may be affected by various procedures of surface treatment applied to the internal tube surface and external bushing surface that come into contact. In some exemplary embodiments, tube 202 is shaped internally with a shape identical to or substantially similar to the shape of the proximal end of a cover such as cover 30 (i.e. proximal end 32). In an exemplary use procedure, the needle with needle bushing 20 is inserted into the tube from top side 206 along axis 204 until the bushing fits tightly in tube 202. The degree of friction between the internal surface of tube 202 and a needle bushing accommodated therein is enough to hold the cannula at substantially 90 degrees to the plane of securing wings 210 when these wings are attached to the body (see FIG. 6 (b)).

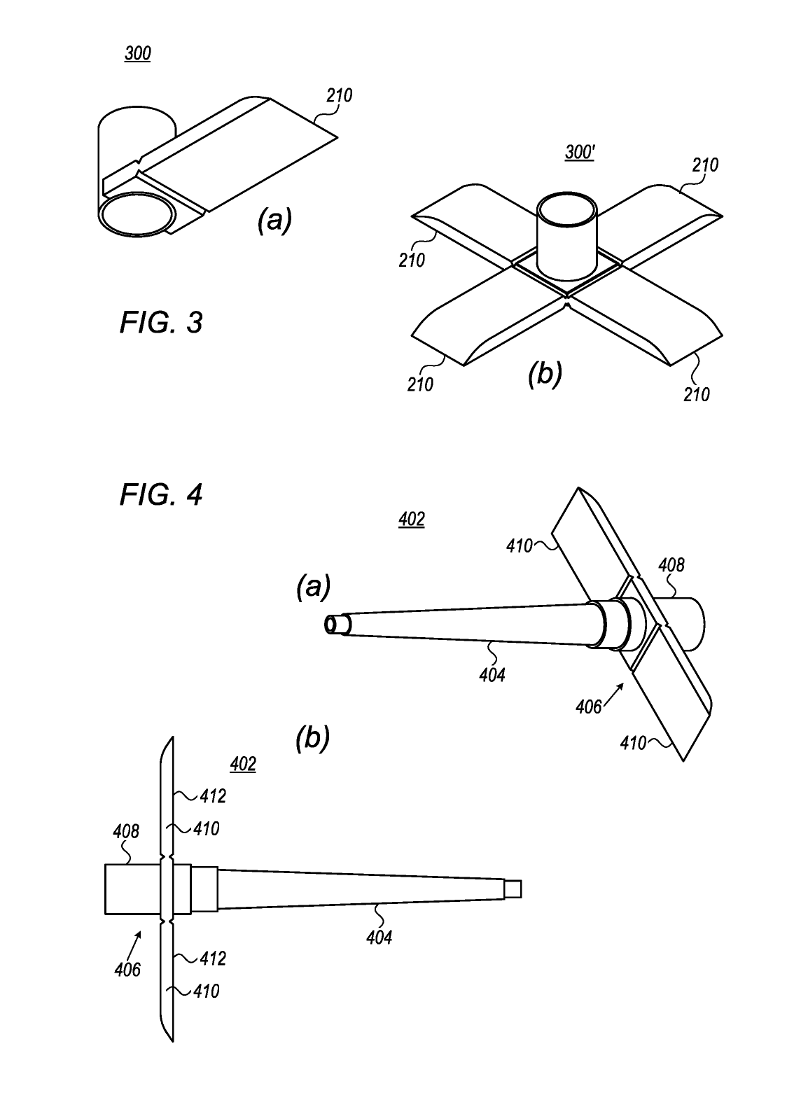

Suitable materials for tube 202 and securing wings 210 may be similar as those used for cover 30, i.e. hard polymeric materials such as thermoplastic polymeric resins, which include polycarbonate, polystyrene, polypropylene and the like. While cannula holder 200 in FIG. 2 is shown as having a securing member in the form of two perpendicular securing wings, the number of securing wings may change. For example, FIG. 3a shows an embodiment of a cannula holder 300 with one wing 210, and FIG. 3b shows an embodiment of a cannula holder 300′ with four wings 210. Other cannula holder embodiments may include for example a securing member in the form of a circular disc or a rectangular plate (not shown) that replaces the wings and has the same securing function.

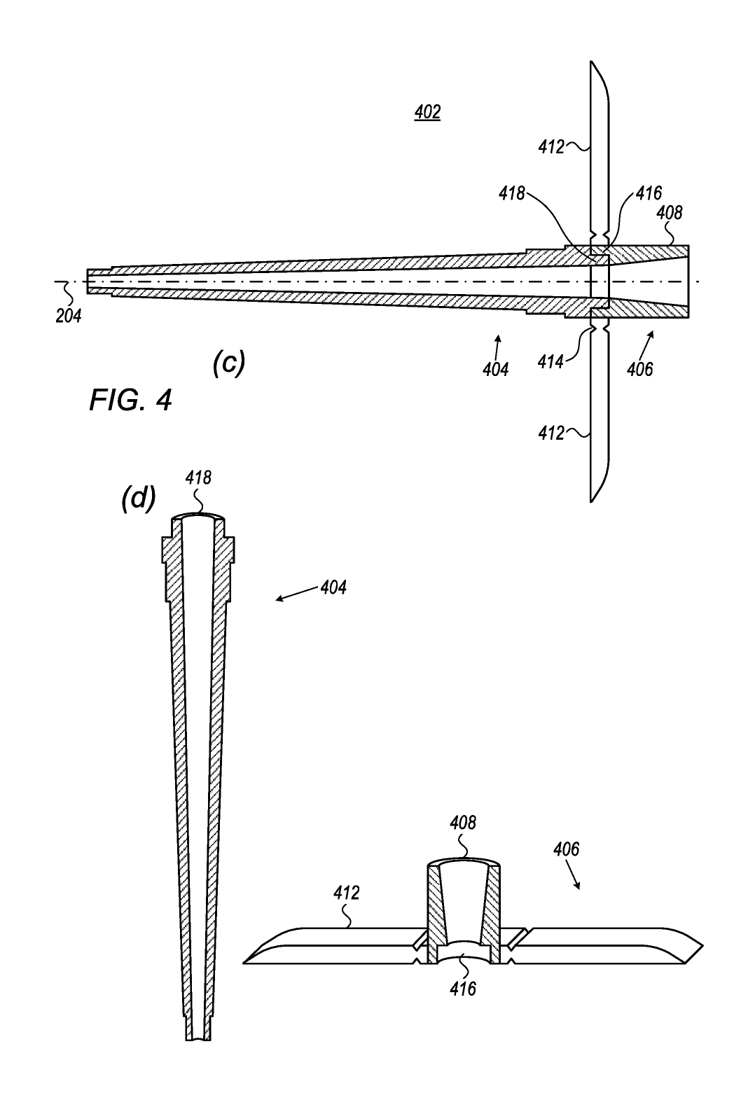

While cannula holder 200 is shown as a self-standing device in FIGS. 2 and 3, in some embodiments a cannula securing device (cannula holder) disclosed herein may be part of, or integrated in, a needle and catheter cover such as cover 30. FIG. 4 shows an embodiment numbered 402 of a needle and catheter cover having an integrated cannula holder in (a) an isomeric view, (b) a side view and (c) a side cross section along a longitudinal axis identical with axis 202 above (and numbered the same). Cover 402 comprises two sections: a distal cover section 404 and a proximal cover section 406, the proximal cover section shaped to serve as a cannula holder similar to cannula holder 200. Like cannula holder 200, proximal cover section 406 includes a tubular section (tube) 408 and perpendicular securing wings 410 with respective contact surfaces (or “attachment surfaces”) 412. Consequently, proximal cover section 406 may serve as a cannula holder (and named then “cannula holder 406”). As shown in the cross sections in FIG. 4 (c) and (d), distal cover section 404 and cannula holder 406 may be separable parts that can be assembled (attached) together and separated (detached, disassembled) through a simple mechanical interface 414 at a plane that coincides with the plane of contact surfaces 412. Since it includes two parts, cover 400 may be also referred to as a “two-part” cover. The mechanical interface may include a cylindrical internal recess 416 in cannula holder 406 that fits tightly with a cylindrical “shoulder” protrusion 418 in a proximal end of distal cover section 404. Other mechanical interfaces that can serve to assemble and detach the two parts of cover 402 would be clear to one of ordinary skill. When provided as a regular cover for the needle and catheter of a cannula, see FIG. 5, two-part cover 400 may easily have distal cover section 404 and holder 406 separated from each other, with cannula holder 406 serving for securing a cannula as described above.

An embodiment of a two-part cover 502 used in a cannula similar to cannula 10 is shown in FIG. 5 (a)-(c). Cover 500 comprises a distal cover section 504 and a proximal cover section 506 that serves as a cannula holder, the cannula holder including a tube 508 and perpendicular securing wings 510 with respective contact surfaces 512. FIG. 5 (a) shows wings 510 extended perpendicularly to axis 202 and ready for use. Wings 510 include grooves 514 that allow wings 510 to bend along in a forward direction (toward the distal end), FIG. 5 (b) and in a backward direction, FIG. 5 (c). The bending may provide easier and simpler packaging. Before use of the cannula for paracentesis, thoracentesis or similar procedures, cover 502 is removed from the cannula, and distal cover section 504 is separated from proximal cover section 506 by simply twisting, pulling or twisting and pulling actions. FIG. 6 illustrates in (a) and (b) an exemplary procedure for securing a cannula to a body organ in a medical procedure that requires the cannula to be secured to the body organ at substantially 90 degrees. In the example, a cannula holder 606 similar to holders 200, 406 or 506, comprising a tube 608 with a longitudinal axis identical with axis 202 above (and numbered the same) and securing wings 610 with contact surfaces 612, is attached to the skin surface 630 of, for example, a patient's body organ (e.g. back or abdomen) 632. Contact surfaces 612 touch the skin. The attachment may be effected using any known medical taping medium (not shown) made of suitable non-allergenic materials. The taping medium secures wings 610 to the skin, FIG. 6 (a). A catheter assembly 634 with a needle bushing 636 is inserted into tube 608 from its top side along axis 202 until bushing 636 is nested securely in tube 608, FIG. 6 (b). The needle may then be removed, leaving the catheter and cannula parts secured to the body organ at the substantially perpendicular position, due to the friction between the bushing and the internal surface of tube 608.

While this disclosure describes a limited number of embodiments, it will be appreciated that many variations, modifications and other applications of such embodiments may be made. For example, not only procedures such as thoracentesis and paracentesis but also other procedures involving cannulae for draining body liquids may advantageously use a cannula holder disclosed herein. For example, other types of cannulae or IV catheters such as intracaths or Yueh needles/catheters that have a needle bushing that can engage a needle cover, a catheter cover, or a needle and catheter cover, may benefit from dividing the respective cover into two, proximal and distal parts (sections), wherein the proximal cover section is structured and adapted to serve as a cannula holder as described herein. In fact, any cover of any fluid drainage needle or catheter in which a needle bushing tightly engages the proximal section of the respective cover may be converted into, or structured as a two-part cover for providing a cannula securing device as described herein. In general, the disclosure is to be understood as not limited by the specific embodiments described herein, but only by the scope of the appended claims.

All references mentioned in this specification are herein incorporated in their entirety by reference into the specification, to the same extent as if each individual reference was specifically and individually indicated to be incorporated herein by reference. In addition, citation or identification of any reference in this application shall not be construed as an admission that such reference is available as prior art to the present application.

What is claimed is:

1. A cannula securing device, comprising:
a) a tubular section having a bottom and shaped internally to engage and hold securely a cannula needle bushing; and
b) a securing member for attaching the cannula securing device to a patient's skin via an attachment surface in an orientation substantially perpendicular to the skin, wherein the attachment surface is substantially co-planar with the tubular section bottom.

2. The cannula securing device of claim 1, wherein the attachment surface is part of at least one perpendicular wing of the securing device.

3. The cannula securing device of claim 2, wherein the at least one perpendicular wing includes two perpendicular wings.

4. The cannula securing device of claim 2, wherein the at least one perpendicular wing includes four perpendicular wings.

5. The cannula securing device of claim 2, wherein each at least one perpendicular wing includes a groove allowing each perpendicular wing to be bent or folded.

6. The cannula securing device of claim 1, wherein the attachment surface is part a disc or a plate.

7. The cannula securing device of claim 1, made of a hard polymeric material.

8. The cannula securing device of claim 7, wherein the hard polymeric material is a thermoplastic polymeric resin.

9. The cannula securing device of claim 1, wherein the cannula securing device is part of a cannula needle and catheter cover.

10. A cannula needle and catheter cover, comprising:
a) a proximal section shaped to serve as a cannula securing device and including a tubular section having a bottom and shaped internally to engage and hold securely a cannula needle bushing, and a securing member for attaching the cannula securing device to a patient's skin in an orientation substantially perpendicular to the skin; and
b) a distal section connectable to and disconnectable from the proximal section.

11. The cannula needle and catheter cover of claim 10, wherein the securing member includes an attachment surface through which the cannula securing device is attached to the patient's skin.

12. The cannula needle and catheter cover of claim 11, wherein the attachment surface is part of at least one perpendicular wing of the cannula securing device.

13. The cannula needle and catheter cover of claim 12, wherein the at least one perpendicular wing includes two perpendicular wings or four perpendicular wings.

14. The cannula needle and catheter cover of claim 12, wherein each at least one perpendicular wing includes a groove allowing each perpendicular wing to be bent or folded.

15. The cannula needle and catheter cover of claim 11, wherein the attachment surface is part of a disc or a plate.

16. The cannula needle and catheter cover of claim 10, made of a hard polymeric material.

17. The cannula needle and catheter cover of claim 16, wherein the hard polymeric material is a thermoplastic polymeric resin.

18. The cannula needle and catheter cover of claim 17, wherein the thermoplastic polymeric resin is selected from the group consisting of polycarbonate, polystyrene and polypropylene.

19. A method for securing a cannula to a body organ, comprising:
a) providing a cannula securing device that includes a tubular section having a bottom, the tubular section shaped internally to engage and hold securely a cannula needle bushing, and a securing member having an attachment surface substantially co-planar with the tubular section bottom;
b) attaching the cannula securing device to a patient's skin via the attachment surface; and
c) inserting the cannula into the body organ through the tubular section such that the cannula needle bushing is engaged and securely held in the tubular section.

20. The method of claim 19, wherein the attaching includes attaching includes attaching the cannula securing device to the patient's skin in an orientation substantially perpendicular to the skin.
